(19) United States
(12) Patent Application Publication
(10) Pub. No.: US 2026/0318901 A1
(43) Pub. Date: Oct. 15, 2026

(54) BIOPHOTONIC PATTERN RECORDING SYSTEMS AND METHODS

(71) Applicants: Taylor Kenerson, Hawthorne, NJ (US); Igor Nazarov, Medford, OR (US)

(72) Inventors: Taylor Kenerson, Hawthorne, NJ (US); Igor Nazarov, Medford, OR (US)

(21) Appl. No.: 19/461,150

(22) Filed: Jan. 27, 2026

Related U.S. Application Data
(60) Provisional application No. 63/758,726, filed on Feb. 14, 2025.

(51) Int. Cl. G01N 33/483; G01N 21/15; G01N 21/17

(52) U.S. Cl. CPC G01N 33/4833; G01N 21/15; G01N 2021/1721

(57) ABSTRACT

The invention is directed to a system comprising an apparatus for recording biophotonic patterns. The apparatus includes a stretched truncated bipyramidal resonant chamber with a circumferential resonant gap. The chamber comprises EMF modulation capabilities between 30 MHz and 6,000 MHz, and an offset CCD sensor system with specialized optical filtering. The system enables extended integration times exceeding 1,000 seconds and provides enhanced pattern stability through controlled standing wave formation and optimized signal-to-noise ratio.

### CROSS-REFERENCE TO RELATED APPLICATIONS

This application claims priority to U.S. Provisional Patent Application No. 63/758,726, filed on Feb. 14, 2025, the entire content of which is hereby incorporated by reference herein.

### FIELD OF THE INVENTION

The present invention relates generally to optical and electromagnetic measurement systems. Specifically, the invention relates to systems and methods for recording biophotonic patterns using a resonant chamber geometry in combination with modulated electromagnetic fields and optical sensor systems. The disclosed system and methods are configured to enhance signal stability and signal-to-noise performance during detection of low intensity photo emissions from samples.

### BACKGROUND OF THE INVENTION

Early work in the field includes high-voltage photography techniques that required direct contact with photographic materials but offered limited integration capabilities. Further, quantitative measurement systems using photomultiplier tubes in dark chambers enabled first quantitative measurements, but were limited to passive detection without external field modulation. The noted systems represented advancement from contact photography to photomultiplier tubes and from single exposure to initial integration time capabilities. Digital signal processing approaches focused on electromagnetic signatures in aqueous systems, advancing from visual observation to digital recording and pattern analysis. Gas discharge visualization techniques further developed digital imaging and computer analysis capabilities, but remained limited by contact requirements and fixed frequency operation. Detection methods have therefore progressed from contact photography through photomultiplier tubes to modern digital sensors. In addition, field applications have evolved from fixed frequency through pulsed fields to swept frequency approaches. Further, signal processing has advanced from visual observation through digital recording to complex pattern recognition. It would therefore be beneficial to provide a system and method of improved biophotonic detection that overcomes the shortcomings of current systems.

### SUMMARY OF THE INVENTION

In some embodiments, the presently disclosed subject matter is directed to an apparatus for recording low intensity photon emission patterns. As used herein, the term “low-intensity photon emission” refers to optical photon emission having an intensity that is below ambient illumination levels and below the detection threshold of conventional imaging systems operating without extended integration. Low-intensity photon emission may include photon flux levels detectable only through the use of light-tight environments, extended optical integration times, and/or high-sensitivity optical sensors. The emissions may be characterized by photon fluxes on the order of single photons up to several thousand photons per square centimeter per second. For example, low-intensity photon emission can include photon flux densities of about 1 photon per square centimeter per second to about 10,000 photons per square centimeter per second (e.g., at least/no more than about 1, 100, 1000, 2000, 3000, 4000, 5000, 6000, 7000, 8000, 9000, or 10,000 photons per square centimeter per second). The classification of an emission as “low-intensity” can be determined relative to the measurement configuration and detection methodology used instead of by an absolute emission magnitude.

Specifically, the apparatus comprises a light-tight resonant chamber formed as a stretched truncated bipyramidal structure. The resonant chamber includes a first truncated pyramidal section and a second truncated pyramidal section positioned base-to-base, and a circumferential resonant gap between the first and second truncated pyramidal sections. The apparatus includes at least one radio frequency reflective interior surface positioned within the resonant chamber (e.g., the reflective surface can be on one or more top walls, bottom walls, side walls, front walls, or rear walls). The apparatus further includes an optical sensor system comprising a charge coupled device (CCD) positioned at a location offset from a central longitudinal axis (e.g., an imaginary reference axis extending generally through the center of the resonant chamber along its longitudinal direction) of the resonant chamber. The apparatus comprises an optical filter positioned between a sample region of the resonant chamber and the CCD, the optical filter being substantially transparent to ultraviolet (10-400 nanometers) and/or infrared radiation (700 nanometers to 1 millimeter) and spaced from the CCD by an air gap. The apparatus includes an electromagnetic signal generator electrically coupled to an electromagnetic field antenna positioned within the resonant chamber, the electromagnetic signal generator configured to deliver a sine-modulated radio frequency signal over a selected frequency range to establish electromagnetic field distributions within the resonant chamber. The apparatus is configured to record photon emission data from a sample positioned within the resonant chamber during an optical integration period. Recording photon emission data includes accumulating photon-induced charge at an optical sensor during the optical integration period and reading out the accumulated charge as data following the integration period.

In some embodiments, each of the first and second truncated pyramidal sections has side walls forming an angle of about 60 degrees relative to a corresponding base surface.

In some embodiments, the circumferential resonant gap has a width of about 1 centimeter.

In some embodiments, the radio-frequency reflective interior surface comprises aluminum, a metallic foil, a layered conductive tape, or a combination thereof. As used herein, a “metallic foil” refers to a thin sheet of electrically conductive metal (e.g., one or more of aluminum, copper, silver, gold, nickel, tin, zinc, steel, titanium, stainless steel, etc.) capable of reflecting radio-frequency energy. As used herein, a “layered conductive tape” refers to a flexible tape structure comprising at least one electrically conductive layer (e.g., a metallic foil, a metallized polymer film, a conductive fabric or mesh, or a conductive coating) and an adhesive layer enabling attachment to a substrate.

In some embodiments, the radio-frequency reflective interior surface exhibits greater than about 90% reflectivity (e.g., more than 90, 91, 92, 93, 94, 95, 96, 97, 98, or 99% reflectivity) at one or more operating radio-frequency ranges. As used herein, “reflectivity” refers to the ability of a material surface to reflect incident electromagnetic energy rather than absorb or transmit the energy. Reflectivity characteristics may be characterized using RF reflectometry techniques (e.g., S-parameter measurements, ASTM E903) or other known electromagnetic measurement methods.

In some embodiments, the electromagnetic signal generator is configured to generate radio-frequency signals over a frequency range of approximately 30 megahertz to 6,000 megahertz (e.g., at least/no more than about 30, 100, 500, 1000, 1500, 2000, 2500, 3000, 3500, 4000, 4500, 5000, 5500, or 6000 megahertz (MHz).

In some embodiments, the electromagnetic signal generator is further configured to produce a frequency sweep having a sweep rate of approximately one octave per minute, as illustrated in FIG. 8. Thus, a frequency sweep may be implemented by progressively varying the radio-frequency signal over time such that the operating frequency increases or decreases by a factor of two over an interval of approximately one minute. The sweep may proceed linearly on a logarithmic frequency scale or according to other suitable sweep profiles, and may be repeated, reversed, or combined with fixed-frequency operation during recording of photon emission data.

In some embodiments, the optical sensor system is configured to support an optical integration time exceeding 1,000 seconds (e.g., at least 1000, 2000, 3000, 4000, 5000, 6000, 7000, 8000, 9000, or 10,000 seconds or more).

In some embodiments, the optical filter comprises fused quartz or fused silica and is substantially transparent to ultraviolet and/or infrared wavelengths. As used herein, “fused quartz” refers to a high-purity, amorphous form of silicon dioxide (SiO₂) produced by melting and solidifying silica without crystallization, resulting in high optical transparency over a broad wavelength range. As used herein, “fused silica” refers to a high-purity, amorphous form of silicon dioxide (SiO₂) produced from synthetic or natural silica by fusion without crystallization, resulting in excellent optical transparency and low impurity content. Fused silica is non-crystalline and is distinct from crystalline quartz materials.

In some embodiments, the air gap between the optical filter and the optical sensor has a thickness in a range of approximately 1 millimeter to 30 millimeters.

In some embodiments, the optical sensor system is positioned within a wall of the resonant chamber.

In some embodiments, the apparatus includes a data acquisition and processing system configured to receive photon emission data from the optical sensor system and to perform digital signal processing and pattern analysis on the received data. The received signals may be digitized, buffered, and/or stored for processing. The data acquisition and processing system further performs digital signal processing, including one or more of noise reduction, background subtraction, signal normalization, temporal or spatial filtering, and signal enhancement, to condition the photon emission data for analysis. The data acquisition and processing system further performs pattern analysis on the processed photon emission data, including identification, comparison, and characterization of spatial, temporal, spectral, or intensity-based features in the photon emission data, in order to detect patterns or correlations associated with the sample.

As illustrated schematically in FIG. 6, photon emission signals detected by the optical sensor system are provided to a data acquisition and processing system, where the signals are digitized, buffered, and stored for processing. The data acquisition and processing system performs digital signal processing operations to condition the detected photon emission data and further performs pattern analysis to identify spatial, temporal, or intensity-based features in the recorded data.

In some embodiments, the electromagnetic field antenna is positioned within an interior region of the resonant chamber at a location corresponding to a region of electromagnetic field interaction. As used herein, a “region of electromagnetic field interaction” refers to a location within the resonant chamber where electromagnetic energy generated by the electromagnetic field antenna is present at a magnitude sufficient to couple with the resonant chamber geometry and/or interact with a sample positioned within the chamber.

In some embodiments, the presently disclosed subject matter is directed to a method of recording low intensity photon emission patterns. As illustrated in FIG. 7, the method includes positioning a sample within a light-tight resonant chamber and introducing a sine-modulated radio-frequency electromagnetic field into the chamber to establish controlled electromagnetic field conditions. Photon emissions from the sample are optically coupled to an optical sensor system during an extended optical integration period, during which photon-generated signals are accumulated. Following the optical integration period, the accumulated photon emission data are read out, digitally processed, and analyzed to identify spatial, temporal, or intensity-based photon emission patterns. The resulting data may be output, stored, or further processed for subsequent analysis. The method steps illustrated in FIG. 7 may be performed in the order shown or in other suitable orders.

In some embodiments, introducing the sine-modulated radio-frequency electromagnetic field comprises generating an electromagnetic signal over a frequency range of approximately 30 megahertz to 6 gigahertz.

In some embodiments, the method includes sweeping the frequency of the electromagnetic signal at a rate of approximately one octave per minute.

In some embodiments, recording the photon emission data comprises integrating signals from the optical sensor system for an integration period exceeding 1,000 seconds.

In some embodiments, optically coupling photon emissions from the sample to the optical sensor system comprises transmitting the photon emissions through an optical filter comprising fused quartz or fused silica.

In some embodiments, the optical filter is positioned at an air gap distance in a range of approximately 1 millimeter to 30 millimeters from the optical sensor system.

In some embodiments, the method includes digitally processing the recorded photon emission data to perform statistical analysis or pattern characterization of the recorded data.

### BRIEF DESCRIPTION OF THE DRAWINGS

FIG. 1 is a perspective view of a resonant chamber for recording biophotonic patterns in accordance with some embodiments of the presently disclosed subject matter.

FIG. 2 is a side plan view of a resonant chamber for recording biophotonic patterns in accordance with some embodiments of the presently disclosed subject matter.

FIG. 3 is a top plan view of a resonant chamber (with the top symmetrical part of the recording chamber removed) for recording biophotonic patterns in accordance with some embodiments of the presently disclosed subject matter.

FIG. 4 is a schematic perspective view illustrating a stretched truncated bipyramidal resonant chamber geometry, including first and second truncated pyramidal sections positioned base-to-base and a circumferential resonant gap therebetween, in accordance with some embodiments of the presently disclosed subject matter.

FIG. 5 is a graphical representation illustrating an example sine-modulated radio-frequency signal, including a radio-frequency carrier modulated by a sinusoidal envelope, in accordance with some embodiments of the presently disclosed subject matter.

FIG. 6 is a schematic block diagram illustrating signal flow and functional components of the biophotonic pattern recording system in accordance with some embodiments of the presently disclosed subject matter.

FIG. 7 is a flowchart illustrating a method of recording low intensity photon emission patterns in accordance with some embodiments of the presently disclosed subject matter.

FIG. 8 is a graph illustrating an example radio-frequency sweep profile in which the signal frequency increases at a rate of approximately one octave per minute in accordance with some embodiments of the presently disclosed subject matter.

### DETAILED DESCRIPTION OF THE INVENTION

For the purpose of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “an element” means at least one element and can include more than one element. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise indicated, all numbers expressing quantities of components, conditions, and so forth used in the specification and claims are to be understood as being modified in all instances by the term “about.” Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the disclosed packages and methods. Thus, the term “about” is used to provide flexibility to a numerical range endpoint by providing that a given value may be “slightly above” or “slightly below” the endpoint without affecting the desired result.

As used herein, the term “and/or” includes any and all combinations of one or more of the associated listed items.

Relative terms such as “below” or “above” or “upper” or “lower” or “horizontal” or “vertical” may be used herein to describe a relationship of one element, layer, or region to another element, layer, or region as illustrated in the drawing figures.

These terms follow standard engineering conventions where “vertical” refers to the direction parallel to gravitational force, with “up” and “above” being opposite to the gravitational force direction and “down” and “below” being in the direction of gravitational force. “Horizontal” refers to directions perpendicular to the gravitational force.

It will be understood that these terms and those discussed above are intended to encompass different orientations of the device in addition to the orientation depicted in the drawing figures.

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

All methods described herein can be performed in any suitable order unless otherwise indicated herein or otherwise clearly contradicted by context. The use of any and all examples, or exemplary language (e.g., “such as”) provided herein, is intended merely to better illuminate the invention, and does not pose a limitation on the scope of the invention unless otherwise claimed. No language in the specification should be construed as indicating any unclaimed element as essential to the practice of the invention.

Unless otherwise defined, all technical terms used herein have the same meaning as commonly understood by one of the ordinary skills in the art to which this disclosure belongs.

The presently disclosed subject matter is directed to systems and methods for detecting and recording low intensity photon emissions, including those commonly referred to as “biophotons” using controlled optical and electromagnetic measurement environments. The term “biophoton” refers to a low intensity photo emission in the ultraviolet (UV), visible, and/or infrared (IR) portions of the electromagnetic spectrum that are associated with biological systems. These emissions are generally characterized by wavelengths in the range of about 200-800 nanometers and low emission intensities of about 10-1000 photons/cm²/second. Biophotons have also been shown to include coherent properties (similar to laser light) and exhibit delayed luminescence following hyperbolic decay patterns. Further, quantum correlation between photons has been exhibited.

Low intensity photon emissions are observed in a variety of biological and non-biological systems. For example, living organisms emit biophotons in the range of about 100-1000 photons/cm²/second. Similarly, plants have been shown to have peak biophoton emissions during growth phases. Cell cultures have also been shown to have emissions of about 10-100 photons/cm²/second. Non-biological materials (such as crystals) have biophoton emission in the range of 1-10 photons/cm²/second. Similarly, structured water has a biophoton emission range of about 5-50 photons/cm²/second. “Structured water” refers to water that has been altered to form clusters of water molecules organized in the multilayered hexagonal structure by applying a subtle magnetic field or light. Further, processed organic materials have biophoton emissions in the range of 10-100 photons/cm²/second. Thus, emissions can be detected from living organisms, plant matter, cell cultures, crystalline materials, aqueous systems, and other samples.

As noted above, the disclosed measurement system is configured to detect and record low intensity photon emissions using controlled optical integration, electromagnetic field modulation and resonant chamber geometries. The term “light frequencies” refers to the frequency components of electromagnetic radiation detected or measured by the system. The system and method described herein are directed to providing stable and repeatable recording of photo emission patterns without limitation to any particular theoretical mechanism or application outcome. As used herein, the term “photon emission patterns” refers to measurable characteristics associated with low-intensity photon emissions that exhibit non-random structure over time, space, and/or frequency when recorded under controlled measurement conditions. The patterns may include spatial distributions of detected photons across a sensor surface, temporal variations in detected photon counts over an integration period, frequency-dependent variations observed during electromagnetic signal modulation, or combinations thereof. The patterns are identified through digital recording and analysis of photon emission data and do not require that individual photons be visually observable by a human observer.

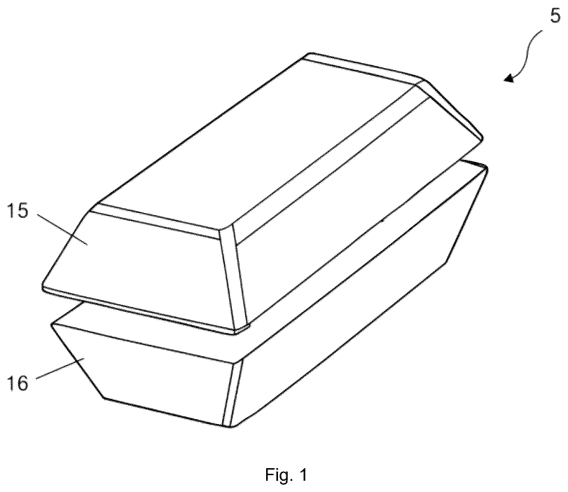

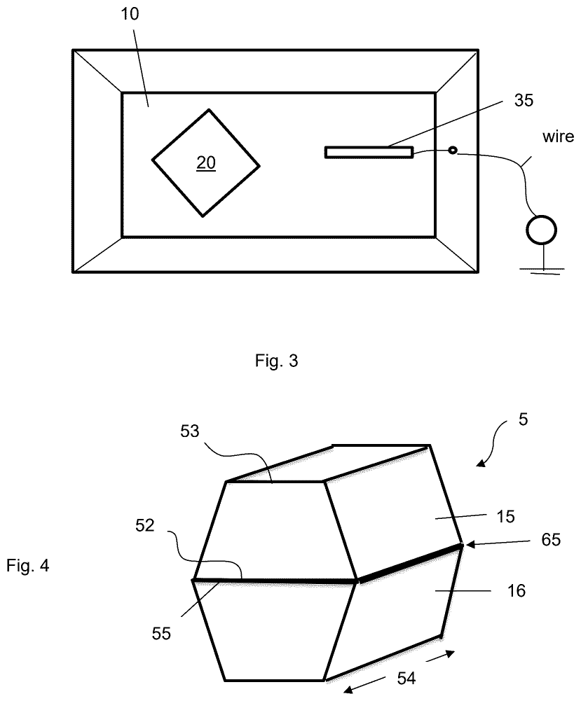

As shown in FIGS. 1-3, apparatus 5 includes resonant chamber 10 comprising two truncated pyramidal sections 15, 16. As used herein, a “resonant chamber” is a bounded structure configured to enhance, stabilize, or selectively amplify electromagnetic or optical energy at one or more characteristic frequencies or wavelengths through resonance effects. As shown in FIG. 4, a “stretched truncated bipyramidal structure” refers to a three-dimensional geometric form 50 derived from opposing pyramidal shapes 15, 16 joined at base 52 (e.g., joined base-to-base) with one or more apexes 53 truncated and the structure elongated along a longitudinal axis 54. Resonant gap 55 is positioned between the first and second sections, as shown. As used herein, a “circumferential resonant gap” refers to a narrow, annular gap formed at an interface between opposing portions of the stretched truncated bipyramidal structure and extending around the perimeter of the structure, the gap being configured to support resonance while the opposing portions remain in overall contact or proximate contact.

Each truncated pyramid is elongated along a longitudinal axis, such that a length of each pyramidal section is greater than its width in some embodiments (e.g., with a length at least two times a width in some instances). As shown, the pyramidal sections are positioned base-to-base to define a stretched truncated bipyramidal resonator with a circumferential resonant gap therebetween. In some embodiments, the gap has a width 65 of about 1 centimeter (e.g., at least/no more than about 0.1, 0.5, 1, 1.5, 2, or 2.5 centimeters), as shown in FIG. 4. As used herein, the “width” of the gap refers to the shortest perpendicular distance between opposing surfaces defining the gap (e.g., between the base of each section 15, 16). Each truncated pyramid can include side walls 11 forming an angle of about 60 degrees relative to a base surface (e.g., at least/no more than about 45, 50, 55, 60, 65, 70, 75, 80, or 90 degrees).

The resonant chamber 10 can be configured as a light-tight enclosure to reduce external optical interference during photon detection. As used herein, a “light-tight” enclosure or chamber is constructed to substantially prevent the ingress of ambient and/or external light while permitting internal electromagnetic or photonic energy to resonate within the chamber for detection or analysis. For example, a light-tight resonant chamber can be configured to isolate low-level photon emissions from ambient light interference so that emission pathways can be detected. It should be appreciated that “light-tight” does not require absolute exclusion of all external photons, but rather sufficient optical isolation to reduce ambient light to a level below the detection threshold of the associated sensor.

The presently disclosed subject matter includes an apparatus to record low intensity photon emission patterns using a combination of extended optical integration times, controlled electromagnetic signal modulation, and specialized optical sensing configurations within an improved resonance chamber. The apparatus is configured to record photon emission patterns from biological and non-biological samples under controlled electromagnetic and optical conditions.

Internal surfaces of chamber 10 can be coated with radio frequency reflective (RF-reflective) material 60. As used herein, “RF-reflective” refers to any suitable material or combination of materials configured to reflect electromagnetic radiation in one or more radio frequency ranges. Suitable RF-reflective materials can include (but are not limited to) aluminum, metallic foils, layered conductive tapes, or combinations thereof. In some embodiments, the RF-reflective material or coating has a reflectivity of greater than about 90% at operating electromagnetic frequencies and substantially lower reflectivity in the visible spectrum (e.g., less than about 15%, 10%, or 5% reflectivity). The geometric configuration of chamber 10 cooperates with the RF-reflective surfaces to support controlled standing wave formation and enhanced resonant field conditions within the chamber during operation.

A sample placement region is defined within resonant chamber 10 at an interior location selected to permit interaction with electromagnetic field distributions established within the chamber. The optical sensor system, including the charge coupled device (CCD), is positioned at a location offset from a central longitudinal axis of the resonant chamber to optimize signal-to-noise performance during photon detection. The offset positioning of the CCD results in the optical sensing axis being displaced from the central longitudinal axis, thereby reducing symmetry-induced resonance artifacts and enabling detection of spatially non-uniform photon emission patterns. The sample placement region may remain centrally positioned within the chamber while the CCD and associated optical path are offset relative to the chamber's central longitudinal axis.

In some embodiments, the offset positioning corresponds to a nodal or anti-nodal region of the electromagnetic signal distribution within the chamber. For example, when a bottom of chamber 10 has a generally rectangular geometry corresponding to two adjacent square regions, positioning a charge coupled device (CCD) sensor at a center of one square region results in an offset relative to the center of the adjacent square region. As used herein, a “charge coupled device” (CCD) is a solid-state optical sensor comprising an array of light-sensitive elements that convert incident photons into corresponding electrical charge signals for image or intensity analysis. Examples of suitable CCDs include low-noise scientific imaging CCDs, including cooled or back-illuminated CCD arrays configured for ultra-low-light detection. The geometric offset positioning enables controlled field interaction and supports repeatable photon detection conditions within the chamber. Stated another way, the CCD is positioned at a location offset from a central longitudinal axis of the resonant chamber in order to detect spatially non-uniform photon emission patterns and reduce symmetry-induced resonance artifacts. The CCD may be offset radially, angularly, or laterally relative to the central longitudinal axis, and the invention is not limited to any specific offset distance.

An industrial high-resolution CCD sensor system 20 is positioned within or adjacent to a wall of resonant chamber 10 and is optically coupled to the sample region through an optical filter 25 (e.g., a quartz-like IR transparent filter, bandpass filters, long-pass filters, short-pass filters, notch filters, neutral density filters, interference filters, absorptive filters, and polarization filters). As used herein, an “optical filter” is a component configured to selectively transmit, attenuate, or block electromagnetic radiation based on wavelength, intensity, polarization, or spectral band. The filter can be positioned between the sample 26 and the CCD sensor 20 with a defined air gap 27 therebetween. In some embodiments, the air gap can have a thickness of about 10 mm (e.g., at least/no more than about 1, 5, 10, 15, 20, 25, or 30 mm). However, the presently disclosed subject matter is not limited and can include any of a wide variety of air gap thicknesses.

In some embodiments, the optical filter can be constructed from a material that is substantially transparent to ultraviolet and/or infrared radiation, such as (but not limited to) fused quartz or fused silica which are glass materials substantially free from additives. As used herein, “substantially transparent” means having sufficient optical transmissivity to permit detection of photon emissions of interest with acceptable signal integrity, for example exhibiting a transmittance of at least about 60%, 70%, 80%, or 90% within a wavelength range of interest, although lower or higher transmittance values may be suitable depending on sensor sensitivity and application context. Optical transmittance values may be determined using standard test methods, such as ASTM E903, ASTM E1164, ASTM E308, or comparable optical transmission measurement standards, incorporated by reference herein. The filter and air gap can be optimized to permit optical transmission while minimally perturbing electromagnetic signal distributions within the resonant chamber.

The CCD sensor system 20 can be configured to support extended optical integration periods, including integration times exceeding 1,000 seconds in some embodiments. Maintaining spatial and geometric stability among the sample region, CCD sensor, optical filter, and electromagnetic signal components contributes to stability of the recorded photon emission patterns during the integration periods. As used herein, an “optical integration period” refers to a defined time interval during which an optical sensor accumulates photon-generated signals prior to readout. During the optical integration period, photon emission events originating from the sample are temporally integrated to produce a composite signal representative of emission intensity and/or spatial distribution.

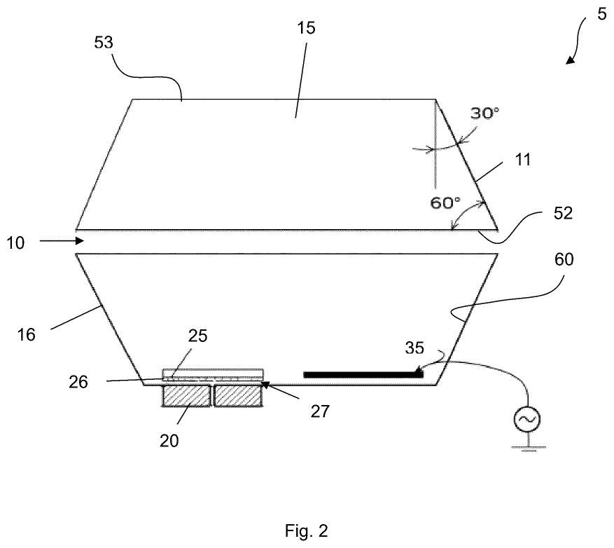

Coupling of electromagnetic fields into the resonant chamber 10 can be achieved through geometric alignment of electromagnetic components relative to field distribution nodes within the chamber. For example, parameters including chamber dimensions, resonant gap dimensions, CCD positioning, and filter spacing can be chosen to correspond to regions of enhanced field interaction. A high frequency electromagnetic signal generator 30 is configured to deliver a frequency modulated electromagnetic signal to the resonant chamber, as illustrated in FIG. 2. As used herein, an “electromagnetic signal generator” is an electronic device configured to generate controlled electromagnetic signals having defined waveform, frequency, and amplitude characteristics. The signal generator 30 is electrically coupled to an electromagnetic field (EMF) antenna 35 positioned within the chamber interior via a wire. As used herein, an “electromagnetic field (EMF) antenna” is a conductive structure configured to transmit, receive, or couple electromagnetic energy between an electromagnetic signal generator and a surrounding space. In some embodiments, the EMF antenna converts an electrical signal generated by the electromagnetic signal generator into an electromagnetic field distribution within the resonant chamber. Examples of suitable EMF antennas include loop antennas, dipole antennas, monopole antennas, coil antennas, patch antennas, and other radiating or coupling structures.

During operation, a sample is positioned in close proximity to the CCD sensor 20 while electromagnetic fields are introduced into the chamber through the antenna 35 to establish controlled field conditions during photon detection.

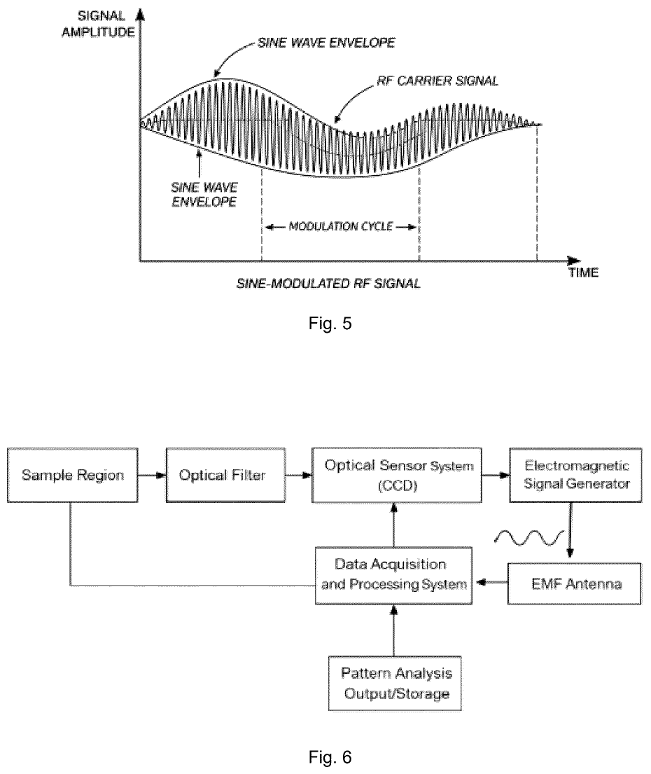

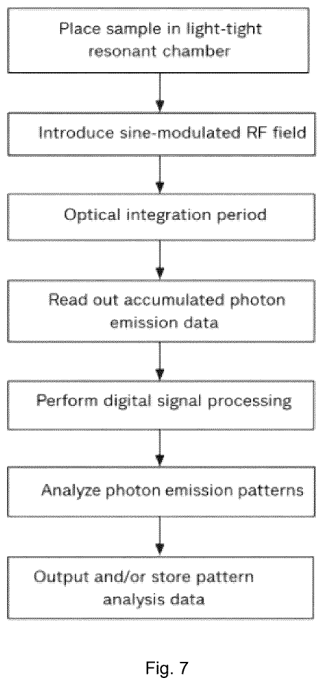

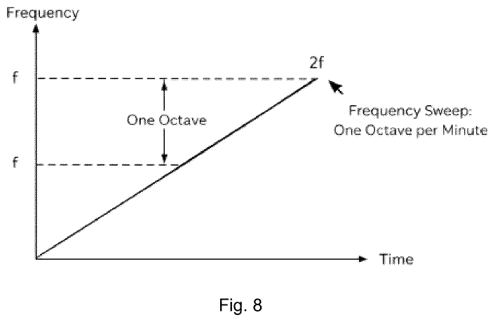

The EMF generation system is configured to provide sine-modulated radio frequency (RF) fields with controlled frequency selection and programmable sweep profiles. As used herein, a “sine-modulated radio-frequency signal” refers to a radio-frequency signal whose amplitude, frequency, or phase is varied according to a sinusoidal waveform. In some embodiments, the sine modulation provides a smooth, continuous variation of the radio-frequency signal to establish stable and repeatable electromagnetic field distributions within the resonant chamber. FIG. 5 illustrates an example sine-modulated radio-frequency signal, showing a radio-frequency carrier modulated by a sinusoidal envelope.

In some embodiments, the EMF generation system is capable of producing frequency sweeps across a predetermined range according to defined sweep rates and modulation parameters. The electromagnetic fields introduced into chamber 10 interact with the chamber geometry to establish standing wave field distributions. The system further includes data acquisition and processing components configured to digitally record signals generated by the CCD sensor system and to perform signal processing operations, such as statistical analysis and pattern characterization of recorded photon emission data. The processing can include analysis of temporal stability, spatial distribution, and repeatability of recorded patterns.

In some embodiments, the electromagnetic signal generator is further configured to produce a frequency sweep having a sweep rate of approximately one octave per minute. As used herein, a “frequency sweep” refers to a controlled variation of signal frequency over time. A sweep rate of approximately one octave per minute means that the signal frequency increases or decreases by a factor of two over a time interval of approximately one minute.

The disclosed measurement system can be used in a variety of analytical contexts. For example, the system can be used to record low intensity photon emission patterns from biological samples and/or non-biological materials. The system can also be useful to evaluate material-dependent emission characteristics under controlled electromagnetic and optical conditions. In addition, the system can be used in industrial or laboratory environments for applications such as comparative analysis, quality assessment, process monitoring, calibration, and long term stability evaluation. The examples are given for illustrative purposes and are not intended to limit the scope of the presently disclosed subject matter.

In some embodiments, the disclosed measurement system can be used in investigative or analytical contexts involving biological sensory systems. For example, photon emission patterns can be recorded from biological samples associated with sensory organs (e.g., taste receptors, olfactory neurons, retinal tissue, or auditory structures) under controlled optical and electromagnetic conditions. Recorded photon emission data can be evaluated for temporal, spatial, or frequency dependent characteristics that vary with different stimuli or sample conditions.

In these applications, the system can be used to observe and compare photon emission patterns associated with different sensory inputs or physiological states without limitation to any particular biological mechanism. For example, emission data recorded from samples associated with gustatory, olfactory, visual, or auditory systems may be analyzed for distinguishing features or pattern variability using digital signal processing and pattern analysis techniques. References herein to quantum, electromagnetic, or other physical phenomena are provided for descriptive context only and are not intended to define or limit the operational principles of the disclosed system.

In some embodiments, the disclosed system may also be used in experimental or research environments to evaluate interactions between electromagnetic field exposure, optical measurement conditions, and photon emission behavior in biological or non-biological samples. Such use may include comparative testing, controlled observation, or exploratory analysis of sample-dependent emission characteristics.

The disclosed system may further be applied in agricultural, environmental, or material analysis contexts. For example, photon emission patterns may be recorded from plant material, seeds, water samples, soils, fertilizers, or other agricultural inputs to support comparative studies, monitoring, or characterization under defined measurement conditions. These example applications are provided for illustrative purposes only and are not intended to limit the scope of the disclosed subject matter or to assert any particular biological outcome or performance result.

Many modifications and other embodiments of the inventions set forth herein will come to mind to one skilled in the art to which these inventions pertain having the benefit of the teachings presented in the foregoing descriptions and the associated drawings. Therefore, it is to be understood that the embodiments of the invention are not to be limited to the specific embodiments disclosed and that modifications and other embodiments are intended to be included within the scope of the invention. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the invention. In this regard, for example, different combinations of elements and/or functions than those explicitly described above are also contemplated within the scope of the invention. Although specific terms are employed herein, they are used in a generic and descriptive sense only and not for purposes of limitation.

### EXAMPLES

### Example 1

### Biophoton Flux Emitted by Plants

A low-intensity light-sensitive setup was tested for the analysis, recording, and storage in electronic format of biophoton flux emitted by various plants. The biophoton flux profiles recorded as a spectral diagram were combined as an electronic file and applied to the other plants through the LED illumination system modulated by the electronic signals derived from the created file. The tested plants were analyzed for changes in mass gain and chemical composition. Preliminary data collected in the laboratory and several greenhouses indicated the feasibility of the method to enhance plants'growth and alter their micro-elemental content.

Plants'emission of biophotons is the extremely weak radiation flux (several hundred photons per second per cm²) in the optical range of the spectrum. It is believed that biophotons are constantly released and absorbed by the cell's DNA, connecting organelles, cells, and tissues, creating a communication network that connects all life processes in the plant. In addition, part of the biophoton flux emitted externally from the leaves broadcasts the state of the plant's well-being. The flux is more intense in healthy-growing plants and gets weaker and less coherent in plants subjected to a disease or stressful environmental conditions, such as dehydration or undernourishment.

Ater the biophoton flux is electronically recorded and transformed into the frequency-modulated current, it applies to the LED lighting devices, creating emission modulations. The modulated LED emission illuminates water through special filters such that the water acquires new qualities (“water structuring.”) The water structuring was confirmed by different methods, such as high-frequency and Raman Spectroscopy, Magnetic Resonance Imaging, and others. It was shown that water structuring leads to changes in the vibrational spectrum of water that ultimately forces the molecular structure of aquatic solutions to attain the higher order of organization and change the way water interacts with living organisms, causing various effects on them.

Preliminary experiments demonstrated biophoton flux patterns transported to the test plants through the water structuring (as compared to the control) improve germination rates for old seeds, facilitate faster growth of seedlings, and increase gain in the resulting biomass for different plants both with and without fertilizers when the same resources are used.
Methods: A three-stage setup was used to accomplish this research. In the first stage, a holographic biophoton pattern resulting from interference of laser light and the plant fluorescence emission was captured by the charged-couple device (CCD). A signal from the CCD was evaluated by the spectrum analyzer, where it was transformed into a digital signal suitable to be used further in the LED illumination system.

The next stage was based on the single photon counting unit combined with a special chamber designed for registering low light emissions. The system allowed for registering biophoton flux from a plant sample, while it was illuminated by different LEDs. A digital signal was used for current modulation of LED. After illuminating a test plant sample with the modulated emission, a delayed fluorescence intensity was recorded. Different modulation patterns were evaluated by comparing the fluorescent intensity of the plant sample before and after LED light processing. As soon as the favorable digital spectrum signature was chosen, it was processed in the closing stage where the test plants were grown in a controlled environment while being illuminated by LED light modulated with the chosen patterns and irrigated by water structured by the same LED emission pattern. After the growing cycle for the test plant was finished, the plant was evaluated for chemical and physical changes.
Results: The results indicated that electronic signals taken from the plants through the biophoton flux can be used for modulating the emission intensity of the LED lighting systems and subsequent water structuring used in agriculture and affect the growth and chemical composition of plants. Several pilot studies were conducted with different agricultural products for getting quantitive results.
Seeds Growing: Two formulas (herein, by formula a holographic combination of several biophotonic patterns taken from different plants is assumed) aiming for plant growth and health were analyzed for fresh hemp seeds. Seeds of two different strains of hemp were grown in a greenhouse-like environment using standard procedures. Results showed that biophoton flux processed plants visually outgrew the control group in the final biomass count, showing a remarkable result for one formula (+58%) and an impressive result for another (+40%).
Plant Growing: To evaluate the possibility of structuring aqueous mixtures other than irrigation water, a study of commercial fertilizer was conducted on growing hemp plants. Two different hemp strains were tested. The plants were given the same amount of fertilizer for the entire growing season, following a standard protocol. Once the plants reached maturity, they were stripped of the associated stem to calculate the total extractable biomass weight. The fertilizer diluted with structured water showed high efficacy for hemp, especially for the strain Special Sauce hemp, yielding biomass over the control of up to 84% (±5%) and total CBD yield (14%±5% per unit weight).

It was determined that old seeds require a higher concentration of structured water in irrigation water compared to fresh seeds. The best results for germination of old rice and onion seeds were observed with a 0.1% concentration of structured water in irrigation water. Watering the seeds using the noted dilution resulted in increased germination in onion seeds by 65% (SD=2.30) and returned a remarkable increase in the germination of rice seeds of 93% (SD=2.14).
Implications/Discussion: Plant growth and development require a constant supply of essential nutrients. So far, while the approach improves plant yield, the nutritional status of the plants remains unclear. The nutrient accumulation in plants will be evaluated. The association genetics will be an approach of choice to identify candidate genes associated with the variation of nutrient accumulation in plants. Such analysis will lead to a better understanding of the action of the transferred biophoton information on nutrient accumulation, and will lay the foundation for genetic improvement of nutrient accumulation in plants grown in altered conditions. Additional statistical data on different plants and a variety of strains will be collected for the creation of a database allowing for more targeted recommendations for the biophoton patterns to be used, the nutrients to be targeted, water and fertilizer consumption to be reduced, and the like.

### Example 2

### Exploring Technologically Mediated Interactions

### Between Plants and Human Consciousness

This example illustrates use of the disclosed system to record, process, and reapply low intensity photon emission patterns derived from plant material, and to evaluate responses associated with subsequent exposure to signals derived from the recorded data.

Using the apparatus described herein, low intensity photon emission data were recorded from plant samples having characteristic biochemical properties. Photon emission patterns were recorded under controlled optical integration conditions while the samples were positioned within the resonant chamber and subjected to modulated electromagnetic field exposure. The resulting photon emission data were digitized, processed, and stored as electronic signal profiles.

In subsequent experiments, the recorded signal profiles were converted into modulated electronic drive signals and used to control an electromagnetic or optical output device. In one implementation, the recorded signal profiles were used to modulate an illumination source, and the modulated output was applied to a carrier medium. The carrier medium was selected to permit physical isolation from the original plant material while retaining exposure to the modulated signal patterns derived from the recorded biophotonic data.

Two groups of human subjects were evaluated under controlled conditions. A first group included subjects with prior exposure history to plant-derived bioactive compounds, while a second group served as a comparative control group without such exposure history. Subjects in both groups were exposed to the carrier medium processed using the modulated signal patterns, without direct administration of plant material or plant-derived chemical compounds.

Observed responses were recorded using qualitative and quantitative assessment methods. The first group exhibited measurable or reportable responses following exposure to the carrier medium processed using the recorded signal patterns, while the second group exhibited reduced or negligible responses under comparable conditions. In additional tests, exposure to carrier media processed using combined signal profiles derived from multiple recording modalities produced distinguishable response patterns compared to exposure to single-modality signal profiles.

These results demonstrate that signal patterns derived from recorded low intensity photon emissions may be captured, processed, transferred, and reapplied using technological systems, and that such processed signals can produce distinguishable effects when applied to a secondary medium under controlled conditions. The observations further illustrate the utility of the disclosed biophotonic pattern recording system for capturing and reproducing complex signal patterns associated with biological samples, without requiring physical transfer of the original biological material.

This example is provided for illustrative purposes only and is not intended to limit the scope of the disclosed systems or methods, nor to assert any particular biological mechanism underlying the observed responses.

What is claimed is:

1. An apparatus for recording low intensity photon emission patterns, the apparatus comprising:
a light-tight resonant chamber formed as a stretched truncated bipyramidal structure, the resonant chamber including:
a first truncated pyramidal section and a second truncated pyramidal section positioned base-to-base;
a circumferential resonant gap between the first and second truncated pyramidal sections;
at least one radio frequency reflective interior surface positioned within the resonant chamber;
an optical sensor system comprising a charge coupled device (CCD) positioned at a location offset from a central longitudinal axis of the resonant chamber;
an optical filter positioned between a sample region of the resonant chamber and the CCD, the optical filter being at least 60, 70, 80, or 90% transparent to ultraviolet radiation, infrared radiation, or both and spaced from the CCD by an air gap; and
an electromagnetic signal generator electrically coupled to an electromagnetic field antenna positioned within the resonant chamber, the electromagnetic signal generator configured to deliver a sine-modulated radio frequency signal over a selected frequency range to establish electromagnetic field distributions within the resonant chamber;
wherein the apparatus is configured to record photon emission data from a sample positioned within the resonant chamber during an optical integration period.

2. The apparatus of claim 1, wherein each of the first and second truncated pyramidal sections has side walls forming an angle of about 60 degrees relative to a corresponding base surface.

3. The apparatus of claim 1, wherein the circumferential resonant gap has a width of about 1 centimeter.

4. The apparatus of claim 1, wherein the radio-frequency reflective interior surface comprises aluminum, a metallic foil, a layered conductive tape, or a combination thereof.

5. The apparatus of claim 1, wherein the radio-frequency reflective interior surface exhibits greater than approximately 90% reflectivity at one or more operating radio-frequency ranges.

6. The apparatus of claim 1, wherein the electromagnetic signal generator is configured to generate radio-frequency signals over a frequency range of approximately 30 megahertz to 6,000 megahertz.

7. The apparatus of claim 6, wherein the electromagnetic signal generator is further configured to produce a frequency sweep having a sweep rate of approximately one octave per minute.

8. The apparatus of claim 1, wherein the optical sensor system is configured to support an optical integration time exceeding 1,000 seconds.

9. The apparatus of claim 1, wherein the optical filter comprises fused quartz or fused silica and is at least 60, 70, 80, or 90% transparent to ultraviolet wavelengths, infrared wavelengths, or both.

10. The apparatus of claim 1, wherein the air gap between the optical filter and the optical sensor has a thickness in a range of approximately 1 millimeter to 30 millimeters.

11. The apparatus of claim 1, wherein the optical sensor system is positioned within a wall of the resonant chamber.

12. The apparatus of claim 1, further comprising a data acquisition and processing system configured to receive photon emission data from the optical sensor system and to perform digital signal processing and pattern analysis on the received data.

13. The apparatus of claim 1, wherein the electromagnetic field antenna is positioned within an interior region of the resonant chamber at a location corresponding to a region of electromagnetic field interaction.

14. A method of recording low intensity photon emission patterns, the method comprising:
positioning a sample within the light-tight resonant chamber of the apparatus of claim 1;
introducing a sine-modulated radio-frequency electromagnetic field into the resonant chamber using an electromagnetic field antenna coupled to an electromagnetic signal generator;
optically coupling photon emissions from the sample to an optical sensor system through an optical filter spaced from the optical sensor by an air gap, the optical sensor being positioned offset from a central axis of the resonant chamber;
recording photon emission data using the optical sensor system during an extended optical integration period while the electromagnetic field is present within the resonant chamber; and
storing the recorded photon emission data for subsequent analysis.

15. The method of claim 14, wherein introducing the sine-modulated radio-frequency electromagnetic field comprises generating an electromagnetic signal over a frequency range of approximately 30 megahertz to 6 gigahertz.

16. The method of claim 15, further comprising sweeping the frequency of the electromagnetic signal at a rate of approximately one octave per minute.

17. The method of claim 14, wherein recording the photon emission data comprises integrating signals from the optical sensor system for an integration period exceeding 1,000 seconds.

18. The method of claim 14, wherein optically coupling photon emissions from the sample to the optical sensor system comprises transmitting the photon emissions through an optical filter comprising fused quartz or fused silica.

19. The method of claim 18, wherein the optical filter is positioned at an air gap distance in a range of approximately 1 millimeter to 30 millimeters from the optical sensor system.

20. The method of claim 14, further comprising digitally processing the recorded photon emission data to perform statistical analysis or pattern characterization of the recorded data.
